SEQUENCE LISTING (8 sequences)

SEQ ID NO: 1 - protein, 23 aa, Artificial Sequence
  Synthetic construct
ACADLDLEALAPYIPADDDFQLR

SEQ ID NO: 2 - protein, 25 aa, Artificial Sequence
  Synthetic construct
ACADLDLEALAHYPYIPADDDFQLR

SEQ ID NO: 3 - protein, 23 aa, Artificial Sequence
  Synthetic construct
ACADLDLEALAPYIPADDDFQLR

SEQ ID NO: 4 - protein, 38 aa, Artificial Sequence
  Synthetic construct
ACADESGLPQLTSYDCEVNAPIQGSRNLLQGEELLRAL

SEQ ID NO: 5 - DNA, 21 bp, Artificial Sequence
  VEGF primer
ccatgaactttctgctdtctt

SEQ ID NO: 6 - DNA, 18 bp, Artificial Sequence
  VEGF primer
atcgcatcaggggcacag

SEQ ID NO: 7 - DNA, 24 bp, Artificial Sequence
  VEGF primer
accgcagctaggaataatggaata

SEQ ID NO: 8 - DNA, 20 bp, Artificial Sequence
  VEGF primer
ctttcgctctggtccgtctt

(19) United States
(12) Patent Application Publication
(10) Pub. No.: US 2010/0004325 A1
(43) Pub. Date: Jan. 7, 2010

(54) Use of Baicalein As Prolyl Hydroxylase 2 Inhibitor

(75) Inventors: Eun Gyeong YANG, Seoul (KR); Hyunju Cho, Sacheon-si (KR); Sunyun Kim, Seoul (KR); Hyunsung Park, Seongnam-si (KR)

Correspondence Address: SUGHRUE MION, PLLC, 2100 PENNSYLVANIA AVENUE, N.W., SUITE 800, WASHINGTON, DC, 20037, US

(73) Assignee: Korea Institute of Science and Technology, Seoul (KR)

(21) Appl. No.: 12/017,535

(22) Filed: Jan. 22, 2008

(30) Foreign Application Priority Data
Jan. 23, 2007 (KR) 10-2007-0007036

(51) Int. Cl. A61K 31/352

(52) U.S. Cl. 514/456

(57) ABSTRACT

The present invention is to prove a new molecular and cellular effect of baicalein, which is selected by a prolyl hydroxylase 2 (PHD2) inhibitor screening method using a compound library. Specifically, the present invention quantitatively analyzed the inhibitory effect of the baicalein against PHD2, confirmed the inhibitory effect against FIH (factor inhibiting HIF), analyzed the HIF (hypoxia inducible factor) protein expression induced by baicalein in a cell, and then, confirmed whether the VEGF is expressed by using reporter assay and ELISA. The above effect of baicalein proves that it can be used for a drug treating ischemic diseases, and, therefore, it can be used for other related diseases.

### FIELD OF THE INVENTION

The present invention relates to a new use of baicalein as prolyl hydroxylase 2 (PHD2) inhibitor which is effective in treating ischemic diseases and other related diseases.

### BACKGROUND OF THE INVENTION

Hypoxia inducible factor-1 (HIF-1) is a protein that plays an important role in various cell responses under a hypoxic condition including coordinated regulation of the expression of genes involved in energy metabolism, vasomotor control, angiogenesis and apoptosis. HIF-1α enhances the expression of vascular endothelial growth factor (VEGF) which is known to promote angiogenesis, and thus it can be used for medical research of local anemia (Semenza, G. L., Curr. Opin. Genet. Dev., 8, 588-594, 1998). Also, HIF-1α is suitable for the treatment of ischemic diseases such as coronary insufficiency, cerebral insufficiency and vascular insufficiency due to its function of accelerating the progress of cell cycle, angiogenesis and cell survival.

In normoxia, the degradation of HIF-1α is stimulated by the binding of VHL (von Hippel-Lindau tumor suppressor) protein with HIF-1α, and the binding between the two proteins is dependent on the hydroxyproline residue located at the 402^{nd} or 564^{th} amino acid of HIF-1α. The interaction between the two proteins is regulated by prolyl hydroxylase (PHD)-mediated hydroxylation of specific proline residue of HIF-1α, wherein oxygen, iron, ascorbate and 2-oxoglutarate are required as cofactors (Masson, N. & Ratcliffe, P. J., Journal of Cell Science, 116, 3041-3049, 2003). PHD2 in particular, plays a key role in maintaining HIF-1 at a perpetually unstable state in normoxia (Berra, E., EMBO J., 22, 4082-4090, 2003). Therefore, artificial inhibition of such enzymes induces a hypoxia-mimic condition which can=provides a way to discover a drug that stimulates HIF-1α activity in normoxia.

The present inventors have developed a method for quantitatively analyzing the interaction between the HIF-1 peptide probe having a fluorescent tag and VBC (von Hippel-Lindau-Elongin B-Elongin C) protein complex, and the method is useful for analyzing the activity of PHD in the hydroxylation of HIF-1α.

Baicalein is a flavonoid extracted from the root of Scutellaria Baicalensis, a traditional Chinese medicinal herb, and it is used for the preparation of various drugs and cosmetics based on its anti-cancer, anti-oxidant, anti-inflammation and anti-radical activities (see KR 1992-0002285 and JP 1995-196490).

Recently, it was reported that baicalein inhibits the activity of 12-lipoxygenase, which is excessively expressed in the tissues of tumor such as prostate cancer and breast cancer (Daotai, N., et al., JBC 281, 18601-18609, 2003).

### SUMMARY OF THE INVENTION

Accordingly, it is an object of the present invention to provide a method for preventing or treating diseases caused by the action of PHD2 by administering baicalein to a subject, the diseases are ischemic diseases, preferably, selected from the group consisting of coronary insufficiency, cerebral insufficiency and vascular insufficiency.

### BRIEF DESCRIPTION OF DRAWINGS

The above and other objects and features of the present invention will become apparent from the following description of the invention taken in conjunction with the following accompanying drawings, which respectively show:

FIG. 1: the fluorescence polarization (FP) values representing the inhibition of PHD2-mediated hydroxylation of F-P564 peptide bound to VBC protein observed for 1,040 compounds in a library;

FIG. 2a: the inhibitory activity of baicalein determined by the change in the fluorescence polarization value while increasing the baicalein concentration;

FIG. 2b: the result of examining whether the change in the fluorescence polarization observed in FIG. 2a is caused by baicalein when F-HyP564 peptide binds to the VBC protein;

FIG. 3a: the result of examining whether the hydroxylation of B-P564 peptide treated with the PHD2 occurs depending on baicalein by mass spectrometry;

FIG. 3b: the result of examining whether the hydroxylation of F-N803 peptide treated with an asparagine hydroxylase (FIH1) occurs depending on baicalein by mass spectrometry;

FIG. 4: the result of examining whether baicalein stabilizes HIF-1α in a specific cell by western blotting;

FIG. 5a: the result of examining whether the HIF-1α stabilized by baicalein binds to hypoxia-response element (HRE) in a specific cell by employing reporter assay and the compensated value with β-galactosidase amount.

FIG. 5b: the result of examining whether the HIF-1α stabilized by baicalein binds to hypoxia-response element (HRE) in a specific cell by employing reporter assay and the compensated value with total protein amount.

FIG. 6a: the result of examining whether baicalein affects the VEGF protein expression in a specific cell by employing RT (Reverse Transcriptase)-PCR; and

FIG. 6b: the result of examining whether baicalein affects to the VEGF protein expression in a specific cell by employing ELISA.

FIG. 7: the result of examining whether baicalein promotes angiogenesis in Chick Chorioallantoic Membrane (CAM).

### DETAILED DESCRIPTION OF THE INVENTION

The present inventors have found through the analysis of the interaction between the fluorescence-labeled HIF-1α peptide and VBC protein that baicalein inhibits the activity of the PHD2. Baicalein can generate a hypoxia-mimic condition by effectively inhibiting PHD2, thereby stabilizing HIF-1α which induces transcription of a gene related to pro-angiogenesis.

Accordingly, the present invention provides a method for preventing or treating ischemic diseases by promoting therapeutic angiogenesis comprising administering baicalein to a mammal.

Hereinafter, the present invention is fully described below.

The present invention provides a method of screening for a compound affecting the interaction between the HIF-1α peptide hydroxylated by PHD2 and VBC protein using fluorescence polarization.

In order to perform said screening, a fluorophore-labeled HIF-1α peptide is prepared. Specifically, a peptide containing 556^{th} to 575^{th} amino acids of HIF-1α (GenBank Accession No.: U22431) known as the binding region to the VBC protein is prepared, and an aminocaproic acid linker is conjugated to the N-terminus of the peptide, followed by tagging fluorescein isothiocyanate (FITC) on the end thereof to obtain fluorescein-labeled HIF-1 peptide (F-P564 peptide). Further, the above procedure is repeated for preparing F-HyP564 peptide which has a hydroxylated proline residue at the 564^{th} amino acid position.

Then, the fluorescein-labeled HIF-1α peptide is hydroxylated by treating PHD2 in the presence of each compound of a compound library.

Subsequently, the reactant is subjected to bind to VBC protein and the fluorescence polarization value is measured by a simple and time-saving fluorescence detector (Victor plate reader). Further, the VBC protein and enzymes used above may be separated and purified by a method using dialysis, ultrafiltration, ion-exchange column chromatography, or reversed-phase high performance liquid chromatography.

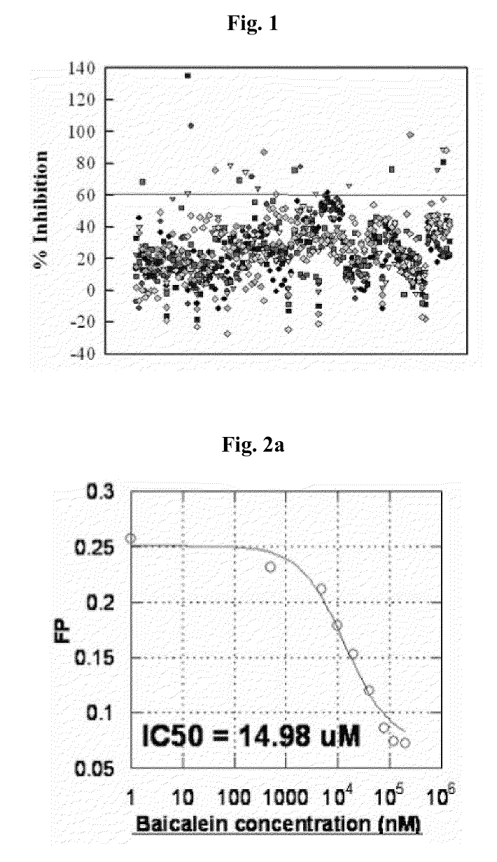

Then, the F-HyP564 peptide, at the same concentration as the F-P564 peptide, is subjected to bind to the same amount of the VBC protein, and the fluorescence polarization value is measured. The observed fluorescence polarization value is used as the positive control to calculate reduction in the fluorescence polarization value, and the overall results are showed in FIG. 1.

As a result of the above screening the present inventors have selected 25 candidate compounds which may potentially inhibit the PHD2 activity.

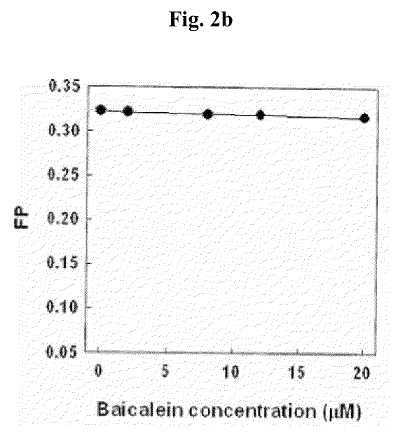

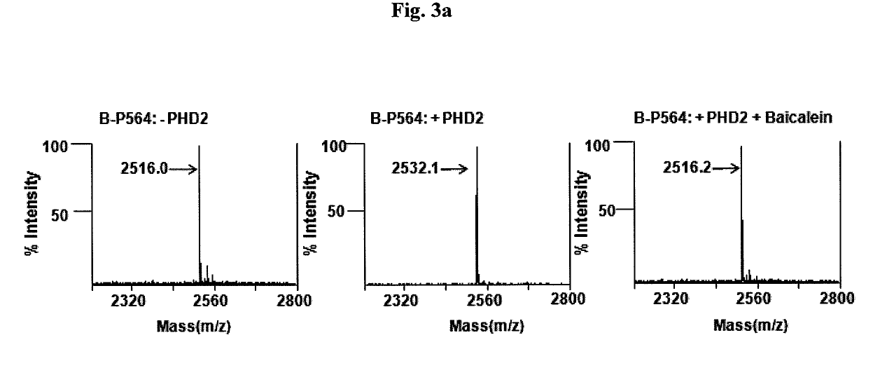

In a further embodiment, the present inventors have performed an experiment which quantifies the degree of the inhibition exerted by baicalein among the candidate compounds and found that the 50% inhibition concentration (IC₅₀) of baicalein is ˜15 μM (see FIG. 2a).

In order to investigate whether such inhibition by baicalein affects only the activity of the PHD2, the fluorescence polarization value of the F-HyP564 peptide bound to VBC protein is measured in the presence of up to 20 μM of the baicalein, and the present inventors have found that baicalein did not affect the interaction of F-HyP564 with the VBC protein per se (see FIG. 2b).

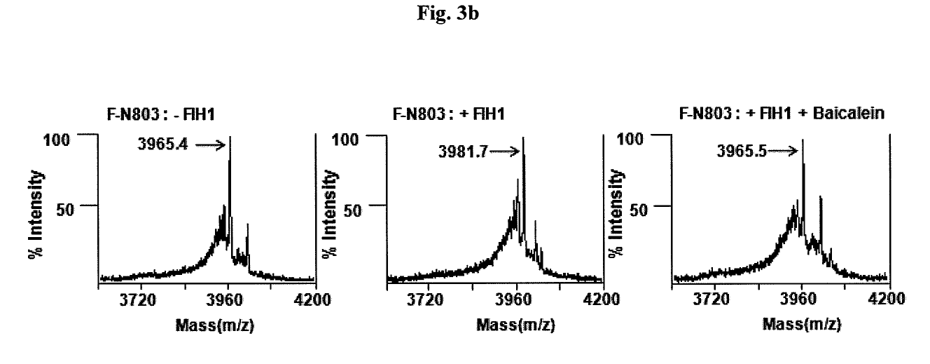

In another preferred embodiment, the present inventors have found that baicalein inhibits the PHD2 activity so that the hydroxylation of B-P564 peptide containing no hydroxyproline residue does not take place (see FIG. 3a).

In a further preferred embodiment, in order to examine whether baicalein inhibits the activity of the FIH1 (factor inhibiting HIF1) enzyme which plays an important role in inhibiting the binding of the HIF-1α to a transcription cofactor CBP (cAMP-response element-binding protein) or p300 protein, a peptide containing the 788^{th} to 822^{nd} amino acid region of the C-terminus of HIF-1α is synthesized and tagged with FITC. The prepared peptide F-N803 is incubated with FIH1 enzyme for asparagine hydroxylation, followed by examination by mass spectrometry, to find that baicalein inhibits the activity of the FIH1 enzyme (see FIG. 3b).

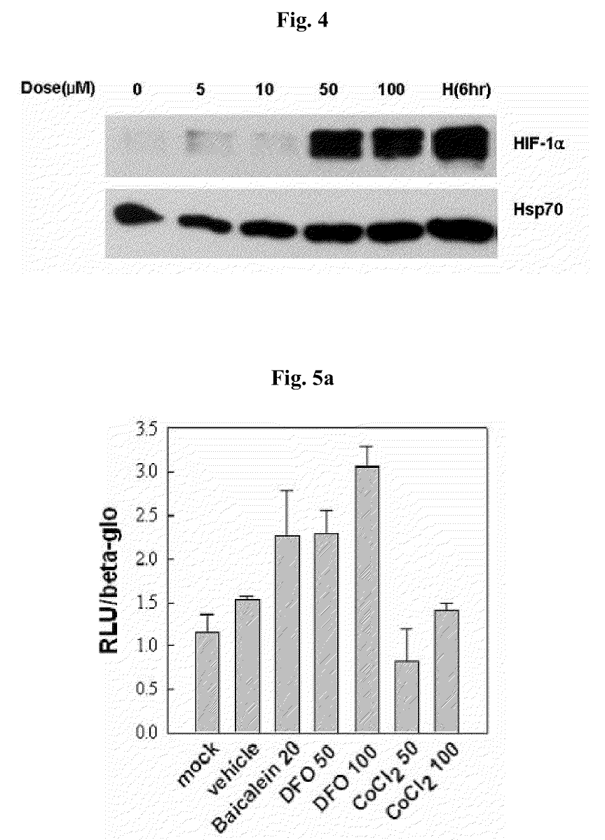

In a further embodiment, in order to examine whether baicalein, which is confirmed to inhibit the activity of the FIH1 enzyme as well as PHD2, stabilizes HIF-1α in a specific cell in normoxia, western blotting is performed, and the result shows that baicalein clearly enhances the stability of HIF-1α by inhibiting the PHD2 activity (see FIG. 4).

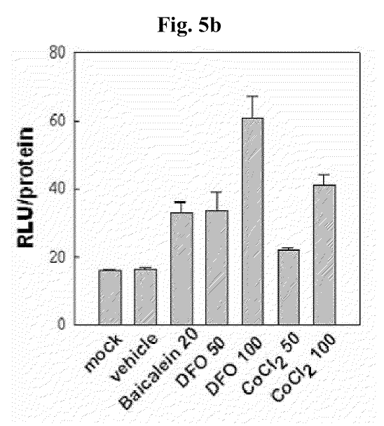

Further examined using a reporter gene is whether the HIF-1α stabilized by baicalein binds in normoxia to hypoxia-response element (HRE) which exists on a target gene promoter. The result shows that the activity of HIF-1α is similar to that in hypoxia-mimic condition, and HIF-1α binds to HRE (see FIGS. 5a and 5b).

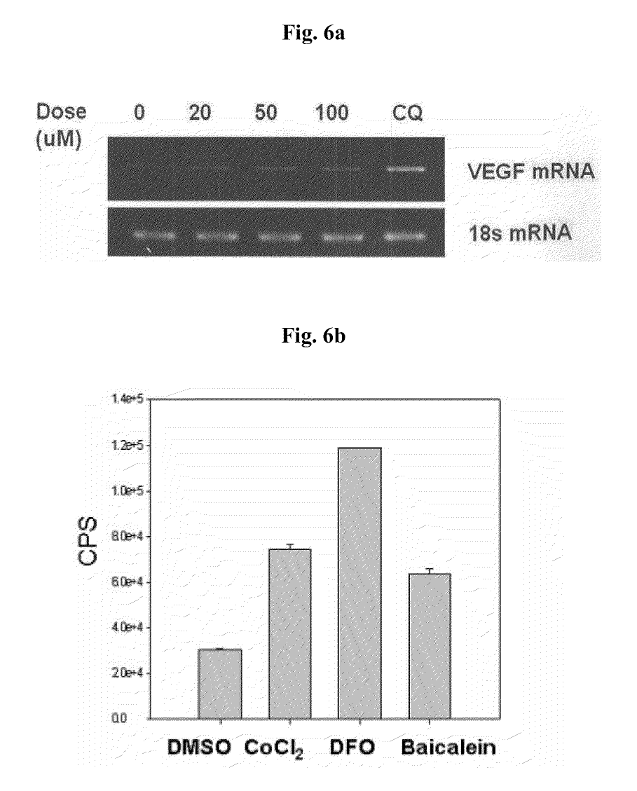

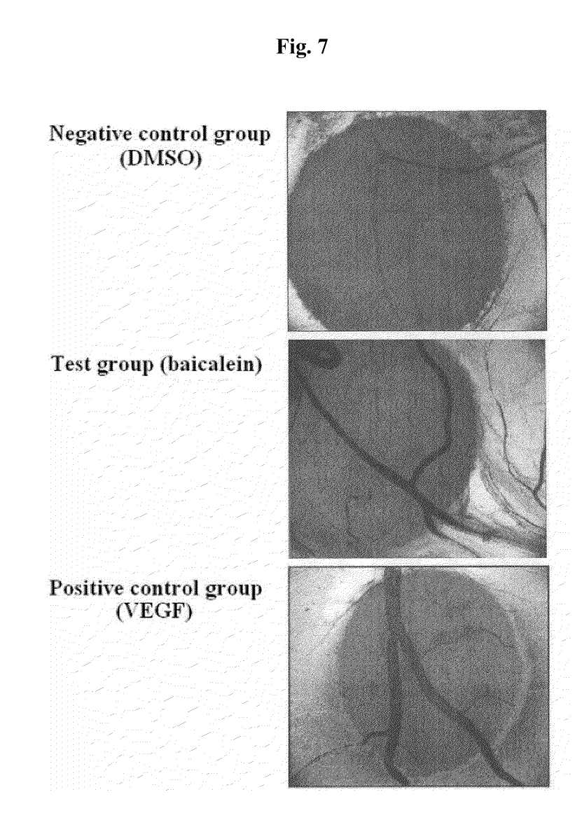

Furthermore, in order to investigate whether the HIF-1α stabilized by baicalein induces the expression of a specific gene by binding to HRE, the secretion amount of vascular endothelial growth factor (VEGF) is measured. The result shows that the amount of the expressed VEGF in the specific cell increases when the baicalein is added (see FIGS. 6a and 6b).

The present inventors have conducted massive screening of 1,040 compounds to identify a potent inhibitor against the PHD activity, and found that baicalein is extremely effective in inhibiting PHD2 activity. Baicalein enhances the target gene expression by promoting the stability of HIF-1α in specific cells.

Accordingly, the baicalein can be used as a medicament to treat or prevent diseases caused by the action of PHD2.

The following Examples are intended to further illustrate the present invention without limiting its scope.

### Example 1

### Preparation of Recombinant Protein

### <1-1> Preparation of VBC and PHD2

In order to prepare VBC protein, the 54^{th}-213^{th} amino acid sequence of human von Hippel-Lindau gene (GenBank accession No.: NM000551) and the 1^{st}-118^{th} amino acid sequence of human Elongin B gene (GenBank accession No.: NM007108) were inserted into pGEX-4T-1 to obtain pGEX4T-VHL-EB expression vector, and the 17^{th}-112^{th} amino acid sequence of human Elongin C gene (GenBank accession No.: NM005648) was inserted into pET29b (Novagen) to obtain pDEV-EC expression vector. These expression vectors were simultaneously transformed into BL21 (DE3) Escherichia coli(E. coli) strain (Novagen) and overexpressed.

Further, in order to prepare PHD2 protein, the 184^{th}-426^{th} amino acid sequence of human PHD2 gene (GenBank accession No.: AJ310543) cloned from human lymphocyte cDNA library was cloned into pGEX-4T-1, and the vector thus obtained was transformed into BL21 (DE3) E. coli strain.

The transformed cells were inoculated to LB medium containing 50 μg/ml of ampicillin and 100 μg/ml of kanamycin (for producing VBC protein) or LB medium containing 50 μg/ml of ampicillin (for producing PHD2 protein), and cultured at 37° C. until OD reached 0.6. Then, the cultured cells were induced to express protein by adding 0.5 mM of IPTG (isopropyl-β-D-thiogalactopyranoside), and further cultured at 18° C. for 15 hours. The cells recovered by centrifugation were resuspended in 10 mM phosphate based saline buffer (PBS, pH 7.4; 110 mM NaCl, 1 mM DTT (dithiothreitol)) supplemented with PMSF (phenylmethylsulfonyl fluoride) and lysozyme at the final concentration of 0.2 mM and 1 mg/ml, respectively, and lysed at 4° C. by ultrasonification.

2% Triton X-100 was added to the cell extract thus obtained, and stirred, which was then put on ice for 10 min, followed by centrifugation for 30 min at 13,000 rpm. 1 mM DTT was added to the separated supernatant, and glutathione (GST)-sepharose 4B resin (Bio-Rad) was mixed thereto and stirred at 4° C. for 2 hours. Phosphate based saline buffer was added to the reacted resin mixture by 10-fold volume, and centrifuged at 2,100 rpm for 5 min to remove supernatant. The above process was repeated three times to remove non-reacted supernatant.

The reacted resin mixture was put in Bio-Spin® Disposable Chromatography Columns (Bio-Rad), filtered by using 5 ml PBS, and filtered again by using 2 ml of 1 M NaCl solution to remove unnecessary reactants. The column was eluted by using 10 mM glutathione (GSH) to obtain GST-VBC and GST-PHD2 proteins. The collected proteins were confirmed by SDS-PAGE and quantified by Bradford protein assay (Bio-Rad).

### <1-2> Preparation of FIH1 Protein

The whole amino acid sequence (1^{st}-349^{th} amino acid) of human FIH1 protein was inserted into pET-28a (Novagen), and the vector thus obtained was transformed into BL21 (DE3) E. coli strain.

The transformed cell was inoculated to LB medium containing 100 μg/ml of kanamycin, and cultured at 37° C. until OD reached 0.6. Then, the cultured cells were induced to express protein by adding 0.5 mM of IPTG (isopropyl-β-D-thiogalactopyranoside), and further cultured at 18° C. for 15 hours. The cells recovered by centrifugation were suspended in cell lysis buffer (50 mM NaH₂PO₄, 500 mM NaCl, 10 mM Imidazole) supplemented with 2-ME (2-mercaptoethanol), PMSF and lysozyme at the final concentration of 0.014 mM, 0.2 mM and 1 mg/ml, and lysed at 4° C. by ultrasonification.

1% Triton X-100 was added to the cell extract thus obtained, and stirred, which was then put on for 10 min, followed by centrifugation for 30 min at 13,000 rpm. Ni-NTA agarose (Quagen) was added to the separated supernatant, and stirred at 4° C. for 1 hour. The resulting solution was washed twice with washing buffer (50 mM NaH₂PO₄, 500 mM NaCl, 20 mM Imidazole), and centrifuged at 2,100 rpm to remove non-reacted supernatant.

The reacted resin mixture was put in Bio-Spin® Disposable Chromatography Columns (Bio-Rad), filtered by using 5 ml washing buffer, and eluted by using 300 mM Imidazole to obtain His-FIH1 protein. The collected protein was confirmed by SDS-PAGE and quantified by Bradford protein assay.

### Example 2

### Screening of PHD2 Inhibitor

### <2-1> Preparation of HIF-1 Peptides

In order to prepare a fluorescent probe of HIF-1α, an aminocaproic acid linker was conjugated to the N-terminus of the 556^{th}-575^{th} amino acid region of the HIF-1α and the very end was tagged with FITC (fluorescein isothiocyanate) to synthesize a target peptide (by Anygen, Korea). The synthesized fluorescent probe was named as ‘F-P564’ peptide having SEQ ID NO: 1. Further, ‘F-HyP564’ having SEQ ID NO: 2, wherein the 564^{th} proline of the F-P564 peptide (the 12^{th} amino acid of SEQ ID NO: 1) was hydroxylated, was synthesized. Further, for mass analysis, a biotin (instead of FITC)-labeled peptide was synthesized and named as ‘B-P564’ having SEQ IN NO: 3. In order to analyze the hydroxylation of the 803^{rd} asparagine residue affecting the transcription activity in the C-terminal region of the HIF-1α, a FITC-labeled peptide was synthesized and named as ‘F-N803’ having SEQ ID NO: 4 (Table 1).

TABLE 1

| Synthesized | |
|---|---|
| peptide | Identification |
| F-P564 | FITC-ACA-DLDLEALAPYIPADDDFQLR |
| (SEQ ID NO: 1) | |
| F-HyP564 | FITC-ACA-DLDLEALAHyPYIPADDDFQLR |
| (SEQ ID NO: 2) | |
| B-P564 | Biotin-ACA-DLDLEALAPYIPADDDFQLR |
| (SEQ ID NO: 3) | |
| F-N803 | FITC-ACA-DESGLPQLTSYDCEVNAPIQGSRNLLQ |
| (SEQ ID NO: 4) | GEELLRAL |

### <2-2> Screening of PHD2 Inhibitor

In order to screen a PHD2 inhibitor, 1040 compounds having biological activities (NINDS Custom Collection II, MicroSource Discovery Systems) were used. First, 1 μl of each compound was aliquoted into a 1.5 ml tube at the final concentration of 20 μM. Then, GST-PHD2 (final concentration: 0.2-0.3 μg/μl) was mixed with the each compound, followed by adding a buffer (20 mM Tris-HCl, pH 8.0, 100 mM NaCl, 1 mM EDTA, 1 mM PMSF, 0.5% NP-40) thereto. Ascorbic acid (final concentration: 200 μM) and α-ketoglutarate (final concentration: 20 μM) were added to the resulting mixture, and then 1 μM of F-P564 peptide was added thereto, followed by reacting at room temperature for 1 hour.

Before completion of the reaction, a buffer (50 mM Tris, 120 mM NaCl, pH 8.0) supplemented with NP-40 (nonidet P-40, final concentration: 0.5%) and 250 nM of the VBC protein were added to the enzyme reactants. After mixing at room temperature, the resulting mixture was transferred to a 96-well plate, and fluorescence polarization was measured by using VICTOR (Perkin-Elmer).

After obtaining the fluorescence polarization value of the mixture of the F-Hy564 peptide (100 nM) containing hydroxyproline and the VBC protein (250 nM) in the buffer by VICTOR, the PHD2 inhibitory activity of each compound was represented in percentage based on that the fluorescence polarization value. 26 compounds with at least 60% of PHD2 inhibitory activity were randomly selected. One of the compounds was excluded because the compound was induced apoptosis when it was treated to cell culture. The other 25 compounds were used to examine the influence on the expression of the HIF-1α and the transcription of a target gene in normoxia.

### Example 3

### Analysis of the PHD2 Activity by Using Baicalein

### <3-1> Analysis of the PHD2 Activity by Measuring Fluorescence Polarization

In order to examine the PHD2 inhibitory activity of baicalein, proline hydroxylation was performed for 20 min with increasing final concentrations of baicalein from 0 to 200 μM. 500 nM of the VBC protein was added to the reactants and mixed. Fluorescence polarization of the resulting mixture was measured by using a fluorometer (Perkin-Elmer), and the size of slit was 6 nm and the integration time was 5 sec. Further, the concentration of baicalein that results in 50% inhibition of the PHD2 activity (IC₅₀) was calculated by using KaleidaGraph program, and the resulting IC₅₀ was 14.98 μM (see FIG. 2a).

Further, in order to confirm that the reduction of fluorescence polarization value by the baicalein, as shown in FIG. 2a, was resulted from the inhibition of the binding of the F-P564 peptide to the VBC protein, F-HyP564 peptide and 500 nM of the VBC protein were admixed in the presence of baicalein at the highest concentration, and fluorescence polarization was measured. Since the fluorescence polarization value did not decrease, it was confirmed that baicalein inhibits the PHD2 activity rather than directly inhibiting the binding of the HIF-1α to the VBC protein (see FIG. 2b).

### <3-2> Confirmation of the PHD2 Inhibitory Activity

For the analysis of proline hydroxylation, 800 μM of baicalein and 1.4 μg/μl of PHD2 were mixed in a buffer (20 mM Tris-HCl, pH 8.0, 100 mM NaCl, 1 mM EDTA, 1 mM PMSF, 0.5% NP-40) supplemented with ascorbic acid (final concentration: 400 μM) and α-ketoglutarate (final concentration: 100 μM). Then, 3 μM of the B-P564 peptide was added to the resulting mixture, and reacted at room temperature for 2 hours. Excess salts in the enzyme reactant were removed by ZipTip_{C18} (Millipore), and the eluted reactant was analyzed by MALDI mass spectrometry. The proline residue was hydroxylated in the baicalein-free reactant, but the residue was not hydroxylated in the reactant treated with baicalein. It indicates that baicalein has the inhibitory activity against PHD2 (see FIG. 3a).

Further, in order to examine the inhibitory activity of baicalein against FIH1, 20 μM of baicalein and 0.55 μg/μl of His-FIH1 were mixed in a buffer (50 mM Tris-HCl, pH 7.5, 5 mM KCl, 1.5 mM MgCl₂) supplemented with ascorbic acid (final concentration: 400 μM) and α-ketoglutarate (final concentration: 100 μM). Then, 4 μM of the F-HIF-1α peptide containing the 788^{th}-822^{nd} amino acid was added to the resulting mixture, and reacted at room temperature for 2 hours. Excess salts in the reactant were removed by ZipTip_{C18}, and the eluted reactant was analyzed by MALDI mass spectrometry. The asparagine residue was hydroxylated in the baicalein-free reactant, but the residue was not hydroxylated in the reactant treated with baicalein (see FIG. 3b). It indicates that baicalein can inhibit not only the PHD2 but also the FIH1, which hydroxylates the specific asparagines residue of HIF-1, thereby inhibiting the binding of p300/CBP protein in normoxia.

### Example 4

### Analysis of the Efficacy of the Baicalein Against HIF-1α

In order to examine the change of the HIF-1α protein expression by baicalein in a cell, HepG2 strain was grown to 80% confluence on a 60 mm tissue culture plate. Baicalein with increasing concentrations was added to the cultured cell and further cultured in normoxia for 6 hours, and as controls, same cell mixtures were cultured in hypoxia. The cells were washed with 4° C. phosphate based saline buffer, and eluted with radioimmunoprecipitation analysis solution containing 150 mM NaCl, 1% Nonidet P-40, 0.5% deoxycholate, 0.1% SDS, 50 mM Tris (pH 7.4). 100 μg/ml PMSF, 10 μg/ml leupeptin, 1 μg/ml antipain, 10 μg/ml aprotinin, 50 mM β-glycerophosphate, 25 mM NaF, 20 mM EGTA, 1 mM DTT and 1 mM Na₃VO₄. Then, the cells were centrifuged, and the cell extracts thus obtained were quantified by Bradford protein assay.

In order to perform the immunoblot against HIF-1α, 20 μg of the cell extract was suspended in SDS sample buffer, and heated in a water bath for 5 min to completely solublize the protein structure. The heated solution was subjected to 8% SDS-PAGE, which has the optimum resolution capacity for the target protein, HIF-1α (100-120 kDa), and each separated gel was transferred to a nitrocellulose membrane. Then, anti-HIF-1α or anti-Hsp70 (BD Pharmingen) as the 1^{st} antibody was treated thereto, and then HRP-conjugated anti-mouse as the 2^{nd} antibody was treated thereto. The membrane was treated with ECL solution and exposed to X-ray film for 5 min, and finally the HIF-1α protein was detected, and subjected to luminescence image analyzer (Model LAS-3000, Fuji) for detailed analysis.

The analysis showed that the HIF-1α protein expression increased in the group treated with more than 50 μM of the baicalein (see FIG. 4), and the increase of the protein expression was similar to that of HIF-1α in hypoxia. Accordingly, it was confirmed that baicalein significantly increases the stability of HIF-1α in normoxia.

### Example 5

### Analysis of the Binding Capacity of HIF-1α with Hypoxia Response Element (HRE)

In order to examine the binding of the HIF-1α stabilized by baicalein in normoxia with HRE existed on a target gene, luciferase assay was conducted as follows. Specifically, HeLa cells (5×10⁴ cells/well) were cultured on a 24-well plate, and 100 ng of p(HRE)₄-luciferase plasmid and 50 ng of β-galactosidase expression vector, pCHO110, were co-transfected thereinto. As a control to 20 μM of baicalein, DFO (deferoxamine) and CoCl₂ were added to the cells, respectively, and cultured for 16 hours. The cell extract was obtained, and the luciferase activity thereof was measured by luminometer (Tuner Designs).

The value was normalized for the β-galactosidase activity value and the total amount of protein measured by BCA protein analysis (Sigma). In case of the β-galactosidase normalization, the group treated with the baicalein showed similar activity value with the group treated with 50 μM of DFO (hypoxia-mimic condition), while the negative control group treated with DMSO showed very low activity value, which indicates that baicalein enhances HIF-1α binding to the HRE (see FIG. 5a). Further, when the value was normalized for the total protein amount, a similar result was obtained (see FIG. 5b).

### Example 6

### Analysis of the Efficacy of Baicalein Against Vascular Endothelial Growth Factor (VEGF)

<6-1> Change of the VEGF mRNA Expression by Baicalein

In order to examine the expression of VEGF known as a representative target gene of the HIF-1α, RT-PCR was conducted as follows. In normoxia, DMSO was treated to HepG2 cells as a negative control, and its influence was observed. As a positive control, HepG2 cells were cultured in hypoxia for 16 hours. In order to amplify cDNA of VEGF mRNA expressed in the cell, the RNA of the cultured cell was subjected to RT-PCR by using forward primer (5′-ccatgaactttctgctdtctt-3′, SEQ ID NO: 5) and reverse primer (5′-atcgcatcaggggcacag-3′, SEQ ID NO: 6). Further, in order to amplify cDNA of 18s rRNA, the RNA of the cultured cell was subjected to RT-PCR by using forward primer (5′-accgcagctaggaataatggaata-3′, SEQ ID NO: 7) and reverse primer (5′-ctttcgctctggtccgtctt-3′, SEQ ID NO: 8) (Table 2).

TABLE 2

| | |
|---|---|
| Primer | Sequence |

| | |
|---|---|
| (SEQ ID NO: 5) | 5′-ccatgaactttctgctdtctt-3′ |
| (SEQ ID NO: 6) | 5′-atcgcatcaggggcacag-3′ |
| (SEQ ID NO: 7) | 5′-accgcagctaggaataatggaata-3′ |
| (SEQ ID NO: 8) | 5′-ctttcgctctggtccgtctt-3′ |

The amplified product was subjected to electrophoresis on 1.5% agarose-gel, and the mRNA expression was quantitated by phosphoimager (Model Las3000, Fuji) (see FIG. 6a).

### <6-2> Change of the VEGF Protein Expression by Baicalein

In order to examine the secretion change of the VEGF protein known as a secretion protein synthesized in a cell, its cell culture solution was subjected to ELISA as follows. Specifically, HeLa cells aliquoted on a 24-well plate were cultured for 24-48 hour, and DMSO, CoCl₂ (200 μM), DFO (125 μM) and baicalein (20 μM) were treated thereto, respectively. After culturing for 16 hours, the cell solution was recovered and centrifuged to separate supernatant. The supernatant was aliquoted on an antibody-coated well plate using human VEGF Quantikine ELISA kit (R&D Systems) to monitor the amount of VEGF in the supernatant. Then, an antibody for VEGF was treated to each well, followed by measuring the amount of the VEGF using luminometer.

As a result, the amount of VEGF was found to significantly increase in the group treated with baicalein compared with that in the control group treated with DMSO, and it was similar to that in the group treated with CoCl₂ inducing hypoxia-mimic condition. Therefore, the stabilization of the HIF-1α by baicalein increases the expression of one of the target proteins, VEGF (see FIG. 6b).

AS described above, fluorescence polarization was used to screen chemicals which inhibit the proline hydroxylation of HIF-1 peptide by PHD, and, as a result, inhibitors which can effectively reduce the activity of PHD were found.

This indicates that baicalein selected according to the subject invention induces hypoxia-mimic condition by stabilizing HIF-1α in normoxia to affect the expression of various target genes.

While the invention has been described with respect to the above specific embodiments, it should be recognized that various modifications and changes may be made and fall within the scope of the invention as defined by the claims that follow.

What is claimed is:

1. A method for preventing or treating diseases associated with inhibition of prolyl hydroxylase 2 comprising administering baicalein to a mammal.

2. The method of claim 1, wherein the diseases are ischemic diseases.

3. The method of claim 2, wherein the ischemic diseases are selected from the group consisting of coronary insufficiency, cerebral insufficiency and vascular insufficiency.
